(19) United States
(12) Patent Application Publication
(10) Pub. No.: US 2012/0053492 A1
(43) Pub. Date: Mar. 1, 2012

(54) TISSUE TREATMENT TOOL AND SIGNAL PROCESSING METHOD USING THE SAME

(76) Inventors: Shuo-Hung CHANG, Taipei (TW); Chung-Han Yang, Taipei (TW); Tsung-Yen Lu, Taipei (TW)

(21) Appl. No.: 13/194,435

(22) Filed: Jul. 29, 2011

(30) Foreign Application Priority Data
Aug. 27, 2010 (TW) 099128771

(51) Int. Cl. A61N 7/00

(52) U.S. Cl. 601/2

(57) ABSTRACT

The present invention provides a tissue treatment tool and signal processing method of the same. The present invention is capable of processing a vibrating signal generated according to a feedback from the soft/hard tissue contacted by the tissue treatment tool during the treatment process so as to obtain an electrical processing signal with respect to a feedback vibration status with respect to soft/hard tissue. According to the electrical processing signal, it is capable of determining the location of the tissue treatment tool or the tissue thickness while treating the target tissue, thereby generating a display signal for displaying the location on a display unit or issuing a stopping signal for stopping providing power to the tissue treatment tool automatically in response to thickness status so as to prevent the soft tissue around the treated tissue from being damaged.

### FIELD OF THE INVENTION

The present invention relates to a tissue treatment technique, and more particularly, to a tissue treatment tool and its signal processing method that are capable of locating a treatment target while simultaneously determining the tissue thickness of the treatment target so as to prevent the peripheral tissue of the treatment target or other internal soft tissue surrounding the treatment target from being damaged mistakenly.

### BACKGROUND OF THE INVENTION

Please refer to FIG. 1A and FIG. 1B, which are schematic diagrams showing a conventional ultrasonic dental treatment device. As shown in FIG. 1A and FIG. 1B, the conventional ultrasonic dental treatment device 2 is configured with a handle 20, which is provided for receiving a driving unit 21 and a treatment tool 22 therein. In addition, the driving unit, being composed of a plurality of piezoelectric elements 210 that are stacking on each other, is coupled to a locking unit 23 that is arranged at the front of the handle 20. As shown in FIG. 1B, the treatment tool 22 is configured with a screw thread connection element 220 at a side thereof, which is provided to connect the treatment tool 22 to the locking unit 23 by screwing so as to enable the treatment tool 22 to receive the vibration signals issued from the driving unit 21 and thus to vibrate accordingly. Consequently, the vibrating treatment tool 22 can be used for removing some unwanted structure from a certain hard tissue, such as a tooth or an alveolar bone, when it is being placed in contact with such hard tissue.

Please refer to FIG. 1C and FIG. 1D, which are schematic diagrams showing the relationship between a sinus cavity and an alveolar bone. As shown in FIG. 1C and FIG. 1D, teeth 13 are surrounded by and anchored to the alveolar bone 10, of jaws and can only grind and chew food normally if they have adequate alveolar bone 10 support. Without enough alveolar bone 10, the jaws cannot support either natural teeth 13 or dental implants if required. There are many reasons for alveolar bone loss, especially for the alveolar bone located corresponding to the sinus cavity 11 as bone recession is very common for such alveolar bone after the diseased teeth anchored thereat are being removed, as shown in FIG. 1D. Consequently, for better anchoring dental implants on the alveolar bone at the area of bone loss, that is usually at the area corresponding to the sinus cavity 11, it is common for a dentist to dill holes on the recessed alveolar bone for enabling a bone grafting procedure to be performed and thus increase the thickness of the alveolar bone. Conventionally, the dentist will use a treatment tool for removing a portion of the recessed alveolar bone at the area corresponding to the sinus cavity 11 so as to construct spaces for the bone grafting procedure. However, during the drill of the alveolar bone using the conventional treatment tool, any careless action from the dentist operating the treatment tool or the dentist is unable to detect the thickness of the alveolar bone during the drilling could lead to a serious consequence that the sinus membrane 12 could be punctured by the treatment tool, causing damages to the surrounding tissues.

Therefore, it is in need of a tissue treatment tool and its signal processing method that are designed for overcoming the aforesaid shortcomings.

### SUMMARY OF THE INVENTION

In view of the disadvantages of prior art, the primary object of the present invention is to provide a tissue treatment tool and signal processing method of the same, adapted for assisting a physician in an operation for treating a target tissue, such as a tooth or a bone, and thereby, assisting the physician to determine the tissue thickness of the treatment target or to determine whether the tissue treatment tool had being placed in contact with the peripheral tissue of the treatment target or other internal soft tissue surrounding the treatment target so as to prevent those tissues other than the target tissue from being damaged mistakenly.

In an embodiment, the present invention provides a tissue treatment device, which comprises: a driving unit, for generating a first vibration signal; a treatment tool, coupled to the driving unit, for receiving the first vibration signal to be used for enabling the same to vibrate while generating a second vibration signal during being used in a treatment; a vibration detection unit, for detecting the first vibration signal and the second vibration signal so as to be driven to vibrate accordingly and thus generating an electrical detection signal; and a signal processing unit, coupled to the vibration detection unit for enabling the same to process the electrical detection signal and thus obtain an electrical processing signal relating to the second vibration signal.

In another embodiment, the present invention provides a tissue treatment device, which comprises: a treatment tool, for receiving a first vibration signal to be used for enabling the same to vibrate while generating a second vibration signal during being used in a treatment; a driving unit, composed of a plurality of transducing elements that are stacking on each other, for generating the first vibration signal and for receiving the second vibration signal so as to enable at least one of the plural transducing elements to generate an electrical detection signal according to the received second vibration signal; and a signal processing unit, coupled to at least one of the plural transducing elements of the driving unit for enabling the same to process the electrical detection signal and thus obtain an electrical processing signal relating to the second vibration signal.

In further another embodiment, the present invention provides a signal processing method for tissue treatment device, comprising the steps of: providing a tissue treatment device, which comprises: a driving unit, a treatment tool, and a vibration detection unit; enabling the driving unit to generate a first vibration signal to be used for controlling the treatment tool to vibrate accordingly; applying the treatment tool to treat a target tissue while enabling the same to generate a second vibration signal to be generated during the treating of the target tissue; enabling the vibration detection unit to detecting the first vibration signal and the second vibration signal so as to be driven to vibrate accordingly and thus generating an electrical detection signal; and processing the electrical detection signal so as to consequently obtain an electrical processing signal relating to the second vibration signal.

Further scope of applicability of the present application will become more apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given herein below and the accompanying drawings which are given by way of illustration only, and thus are not limitative of the present invention and wherein:

FIG. 1A and FIG. 1B are schematic diagrams showing a conventional ultrasonic dental treatment device.

FIG. 1C and FIG. 1D are schematic diagrams showing the relationship between a sinus cavity and an alveolar bone.

FIG. 2A is a schematic diagram showing a tissue treatment device according to an embodiment of the present invention.

FIG. 2B is an exploded view showing a driving unit, a treatment tool and a vibration detection unit that are configured in a tissue treatment device according to the present invention.

FIG. 3 is a schematic diagram showing the use of a treatment tool of the present invention for treating an alveolar bone at an area corresponding to a sinus cavity.

FIG. 4A and FIG. 4B are two schematic diagram showing respectively two tissue treatment devices according to further two embodiments of the present invention.

FIG. 5 is a flow chart depicting the steps performed in a signal processing method for tissue treatment device according to the present invention.

FIG. 6A is a schematic diagram showing an electrical detection signal generated in the present invention.

FIG. 6B is a schematic diagram showing an electrical processing signal generated in the present invention.

### DESCRIPTION OF THE EXEMPLARY EMBODIMENTS

For your esteemed members of reviewing committee to further understand and recognize the fulfilled functions and structural characteristics of the invention, several exemplary embodiments cooperating with detailed description are presented as the follows.

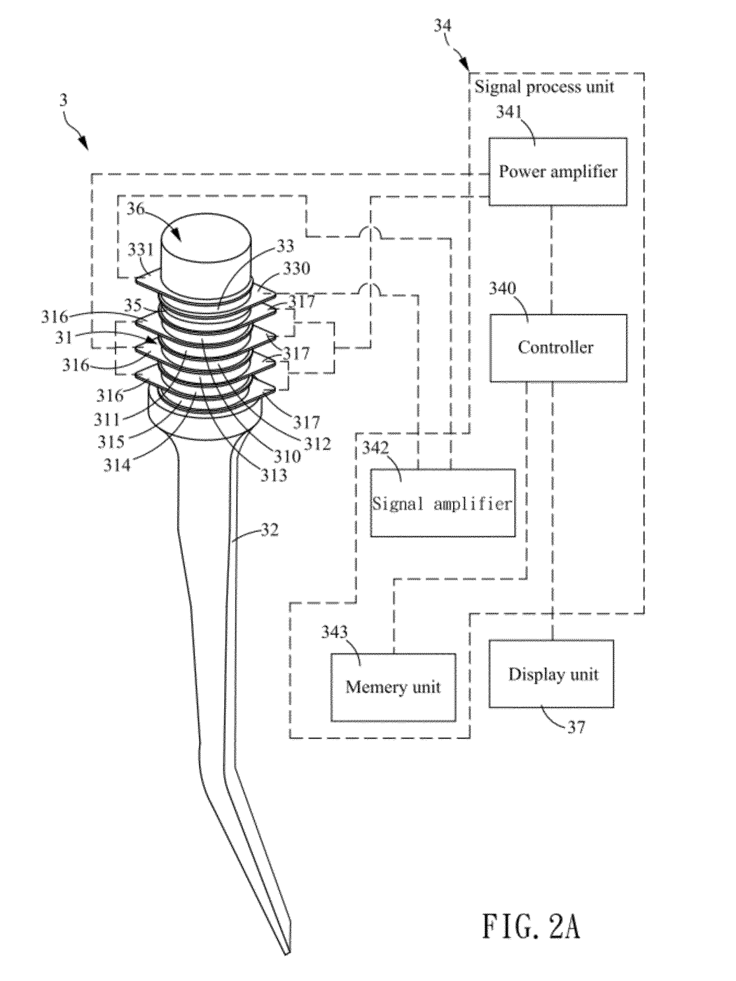

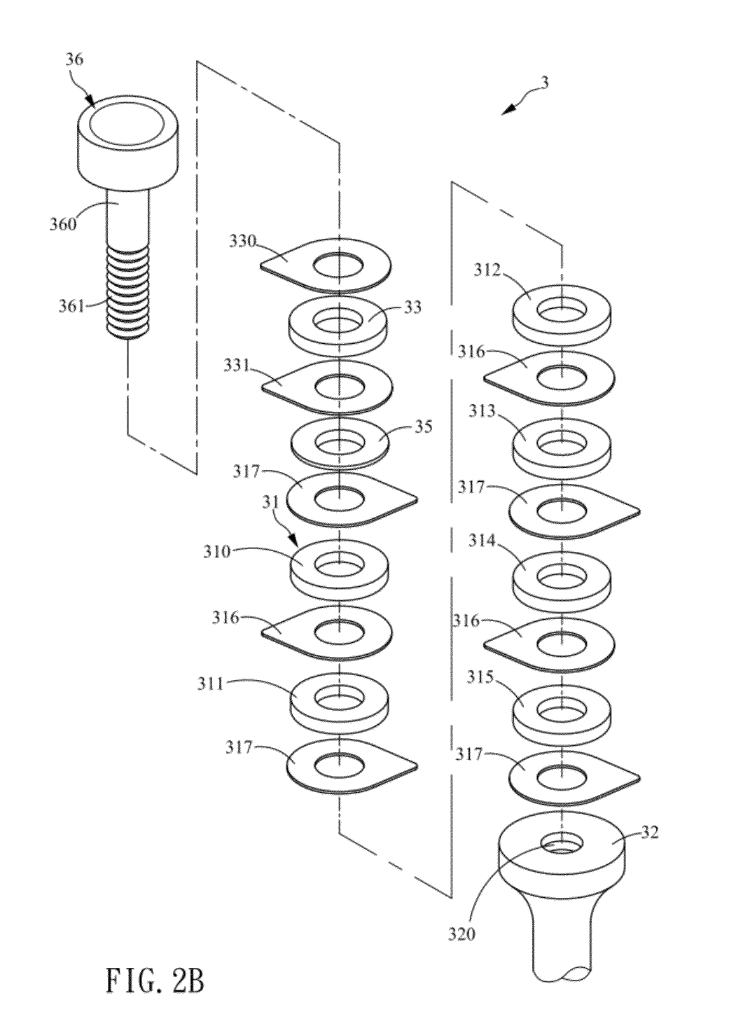

Please refer to FIG. 2A and FIG. 2B, which are respectively a schematic diagram showing a tissue treatment device according to an embodiment of the present invention, and an exploded view showing a driving unit, a treatment tool and a vibration detection unit that are configured in a tissue treatment device according to the present invention. As shown in FIG. 2A and FIG. 2B, the tissue treatment device 3 is composed of a driving unit 31, a treatment tool 32, a vibration detection unit 33 and a signal processing unit 34. In this embodiment, the driving unit 31, being used for generating a first vibration signal, is composed of a plurality of transducing elements that are stacking on each other, as the elements 310˜315 shown in FIG. 2A and FIG. 2B. It is noted that each of the plural transducing elements can be a piezoelectric element, but is not limited thereby. In addition, there is a cathode plate 316 being sandwiched between the transducing elements 310 and 311, and that is also true for the transducing elements 312 and 313, the transducing elements 314 and 315. On the other hand, there are anode plates 317 being disposed respectively at locations that is on the transducing element 310, sandwiched between the transducing elements 311 and 312, sandwiched transducing elements 313 and 314, and sandwiched between the transducing element 315 and the treatment tool 32. Moreover, the amount of such transducing elements required is dependent upon how larger the magnitude of the first vibration signal should be, and thus it is not limited by those shown in FIG. 2A and FIG. 2B.

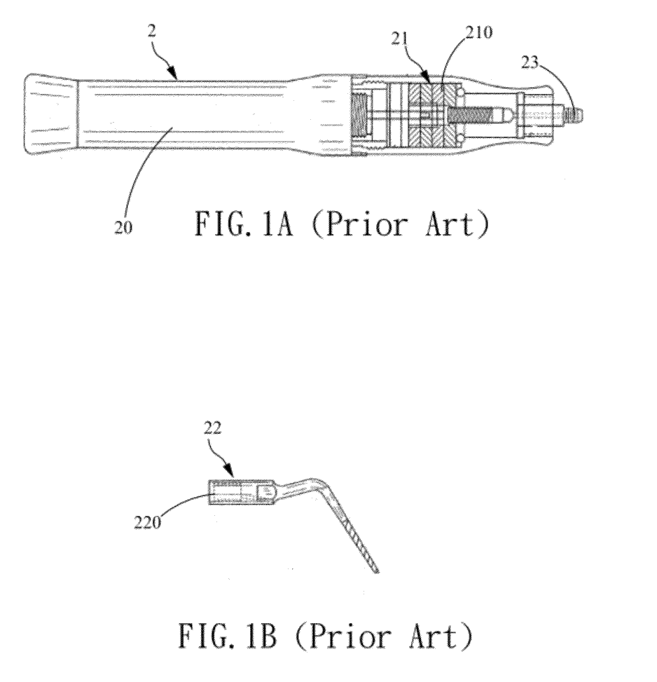

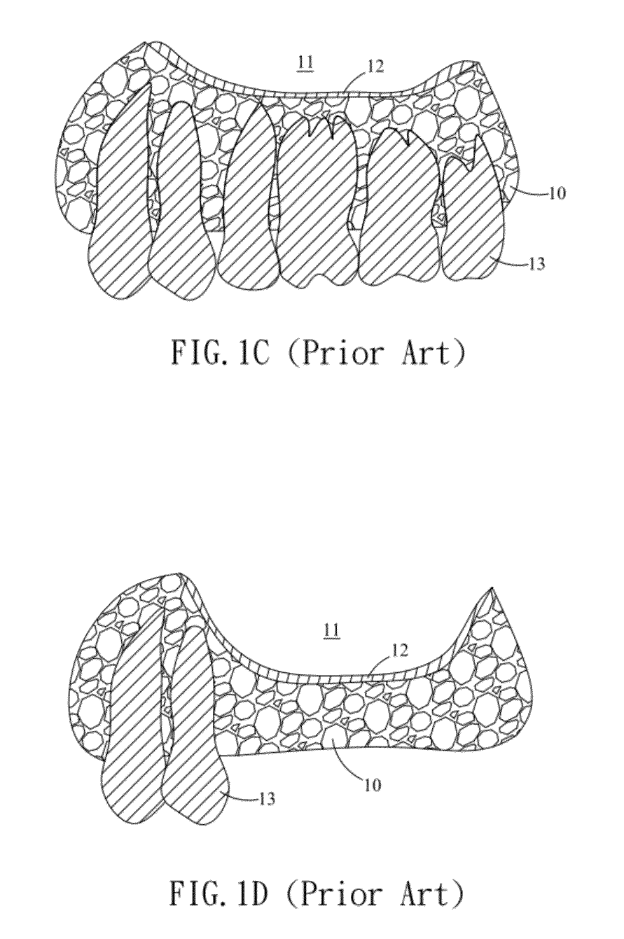

The treatment tool 32 is coupled to the driving unit 31 for receiving the first vibration signal to be used for enabling the same to vibrate while generating a second vibration signal during being used in a treatment. It is noted that there is no restriction relating to the structure of the treatment tool 32 whatsoever, only if it is capable of being driven to vibrate by the first vibration signal of the driving unit 31 and thus used to treat a target tissue. Thereby, the structure of the treatment tool 32 is not limited by the present embodiment. For clarity, the target tissue that is to be treated by the treatment tool 32 of the present invention can be a bone tissue, such as the alveolar bone shown in FIG. 1D, any limb bones, or even teeth. Consequently, the second vibration signal that is being generated by the treatment tool 32 while being used in a treatment can be varied with the variation of the tissue that is being treated, as it is substantially a vibration signal that is generated in response to the different feedbacks corresponding to the different tissues that are being treated by the treatment tool 32. For instance, the second vibration signal generated when the treatment tool is engaging with a sinus membrane of sinus cavity is different from the second vibration signal generated when the treatment tool is engaging with a tooth, and that is also true for gums, alveolar bones, or soft tissues inside a mount, and it will be different even between the treatment of the outer layer of a tooth and the inner tissue of the same tooth, and between the outer formation of a bone and the inner tissue of the same bone.

The vibration detection unit 33 is for detecting the first vibration signal and the second vibration signal so as to be driven to vibrate accordingly and thus generating an electrical detection signal. In this embodiment, the vibration detection unit 33 is a transducing element, but is not limited thereby. As shown in FIG. 2A and FIG. 2B, there are an anode plate 330 and a cathode plate 331 being connected respectively to the two sides of the vibration detection unit 33, and thereby, the electric signal relating to the vibration of the vibration detection unit 33 that is caused by the first vibration signal and the second vibration signal can be received by the anode plate 330 and the cathode plate 331. Moreover, in the present embodiment, the vibration detection unit 33 is being arranged at a side of the driving unit 31, and thus for preventing any short circuit between the vibration detection unit 33 and the driving unit 31, there is an insulation layer 35 being arranged between the two.

In addition, in the embodiment shown in FIG. 2A and FIG. 2B, the driving unit 31 is coupled to the treatment tool 32 through a locking element 36, which is substantially a rod 360 with a threaded end 361 formed at an end thereof. Thereby, the coupling can be achieved by boring the rod 360 through the vibration detection unit 33, the insulation layer 35 and the driving unit 31 so as to screw the threaded end 361 into the screw hole 320 of the treatment tool 32, and thus, the treatment tool 32 is abutted against the driving unit 31 by a side surface thereof so as to enable the treatment tool 32 to received the first vibration signal of the driving unit 31 and thus being driving to vibrate accordingly. The signal processing unit 34 is coupled to the vibration detection unit 33 for enabling the same to receive and process the electrical detection signal and thus obtain an electrical processing signal relating to the second vibration signal.

In FIG. 2A, the signal processing unit 34 is composed of a controller 340, a power amplifier 341 and a signal amplifier 342, whereas the controller 340 is coupled respectively to the power amplifier 341 and the signal amplifier 342. Moreover, as the power amplifier 341 is further coupled to the anode plate 317 and the cathode plate 316 of the driving unit 31, the controller 340 is able to control the magnitude of the power that is being fed to the driving unit 31 by the power amplifier 341, and thereby, further control the magnitude of the first vibration signal. In addition, as the signal amplifier 341 is further coupled to the anode plate 330 and the cathode plate 331 of the vibration detection unit 33, the electrical detection signal generated by the vibration detection unit 33 according to the first and the second vibration signals can be transmitted to the signal amplifier 342 where the electrical detection signal is being amplified and then transmitted to the controller 340 for signal processing so as to obtain the electrical processing signal.

Moreover, the signal processing unit 34 further comprises a memory unit 343, which is used for storing a sample data relating to the signal feedbacks of the treatment tool 32 generated when treating all kinds of different tissues, such as teeth, alveolar bones, limb bones or soft tissue inside a mouth, or even muscles and internal organs. Thereby, the controller 340 compare the electrical processing signal with the sample data stored in the memory unit 343 so as to determine the location of the treatment tool or the tissue thickness of a target tissue based upon the comparison. It is noted that the aforesaid sample data is substantially the second vibration signals that are generated in response to the different feedbacks corresponding to the different tissues that are being treated by the treatment tool 32. Generally, taking the sinus cavity 11 shown in FIG. 1C for instance, there is a sinus membrane 12 located at the interface between the alveolar bone 10 and the sinus cavity 11. Moreover, for a mount cavity, there are soft tissues, such as gums, and inner wall of the mouth cavity, and there are hard tissues, such as teeth, and in detail, each tooth can further be composed of different tissues including enamel, dentin and pulp cavity. Thus, the second vibration signal generated from the treatment tool 32 can be varied with the variation of the tissues that is being treated by the treatment tool 32 as the feedbacks corresponding to the different tissues that are being treated by the treatment tool 32 are different. Nevertheless, the tissues that are being treated can respond differently with the use of different treatment tools 32, i.e. different treatment tools can generated different feedback signals while treating the same target tissue. Thus, a database can be established that contains all kinds of second vibration signals that are generated by different treatment tools while being used for treating various target tissues, and is provided to be stored in the memory unit 343. Consequently, as soon as the control 340 obtains the electrical processing signal, it will compare the electrical processing signal with the sample data stored in the memory unit 343 so as to determine the type of tissue that is currently being treated by the treatment tool 32, no matter it is a sinus membrane, a alveolar bone, a gum, an enamel, a dentin or a pulp cavity. Moreover, the memory unit can be storage media, such as a non-volatile memory, a volatile memory, or a hard disk.

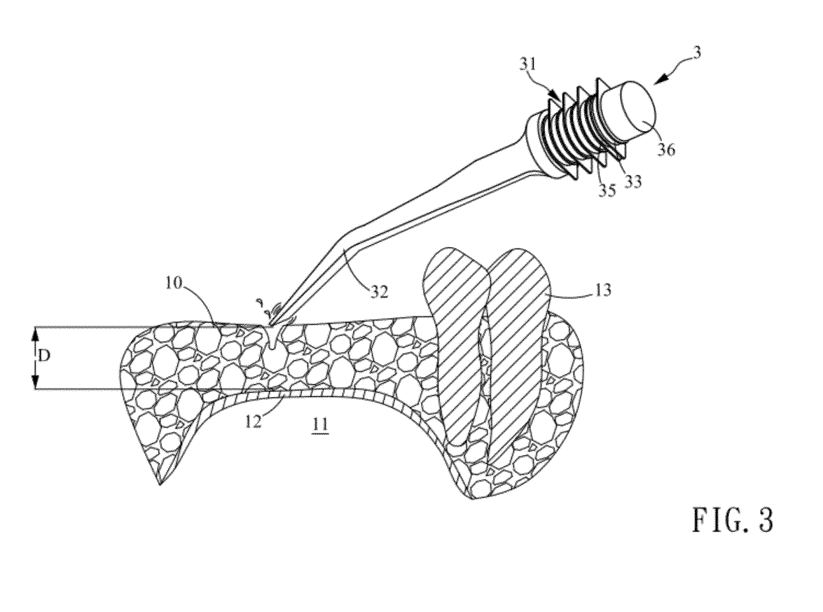

Please refer to FIG. 3, which is a schematic diagram showing the use of a treatment tool of the present invention for treating an alveolar bone at an area corresponding to a sinus cavity. As shown in FIG. 3, the thickness D of the alveolar bone 10 will be varying with the cutting of the treatment tool 32, and consequently, with the thickness variation in the alveolar bone 10, the second vibration signal of the treatment tool 32 that is generated resulting from the feedback of the alveolar bone 10 will be varied. Therefore, it is important to include all kinds of varying second vibration signals that are generated corresponding to the thickness variation in different target tissues into the sample data stored in the memory unit 343. Thus, a dentist operating the treatment tool 32 for treating the alveolar bone 10 is able to determine the thickness of the alveolar bone 10 according to the electrical processing signal. Back to FIG. 2A, operationally when the controller 340, based upon the electrical processing signal, determines that the treatment of the treatment tool 32 upon a hard tissue, such as an alveolar bone or a tooth, had already shaping the treated tissue to a specific thickness, the controller 340 will be enabled to issue a trigger signal to a display unit 37 for enabling the same to generate and display an alert signal to the physician operating the treatment tool 32 and thus informing the physician that the specific thickness of the target tissue had already been reached. It is noted that the display unit can be an audio device for issuing an alarm or a video device for displaying warning icons or lights, and moreover, the video device can be a liquid crystal display device, a light emitting diode (LED) display device, or a LED light fixture with light indication, and so on. In addition, when the controller 340, based upon the electrical processing signal, determines that the treatment tool 32 had engaged with tissues other than the target tissue, such as the soft tissue in a mount cavity, the controller 340 will immediately stop providing power to the driving unit 31.

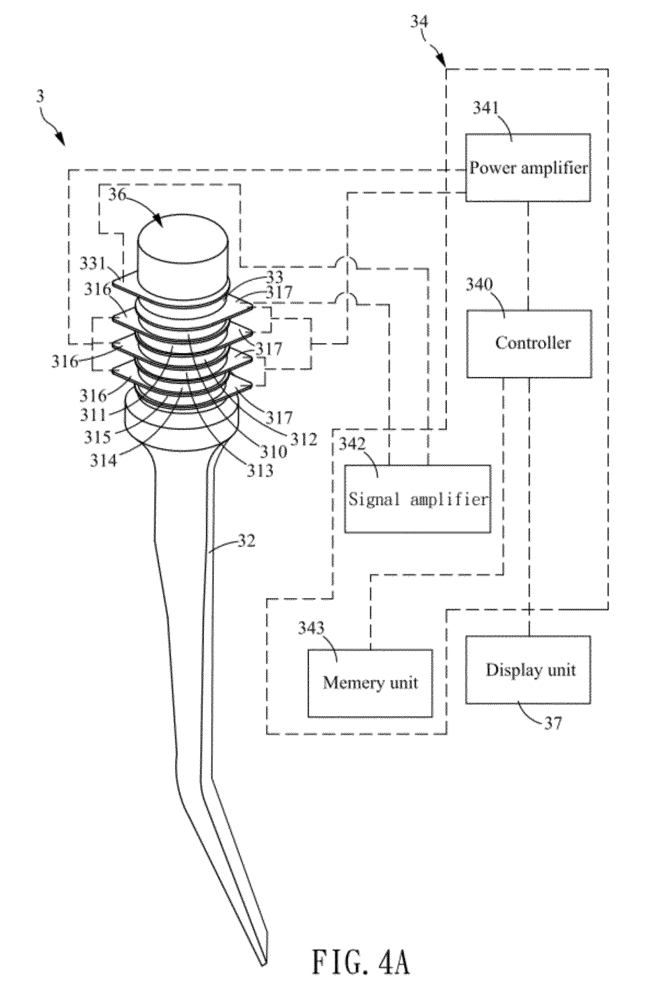

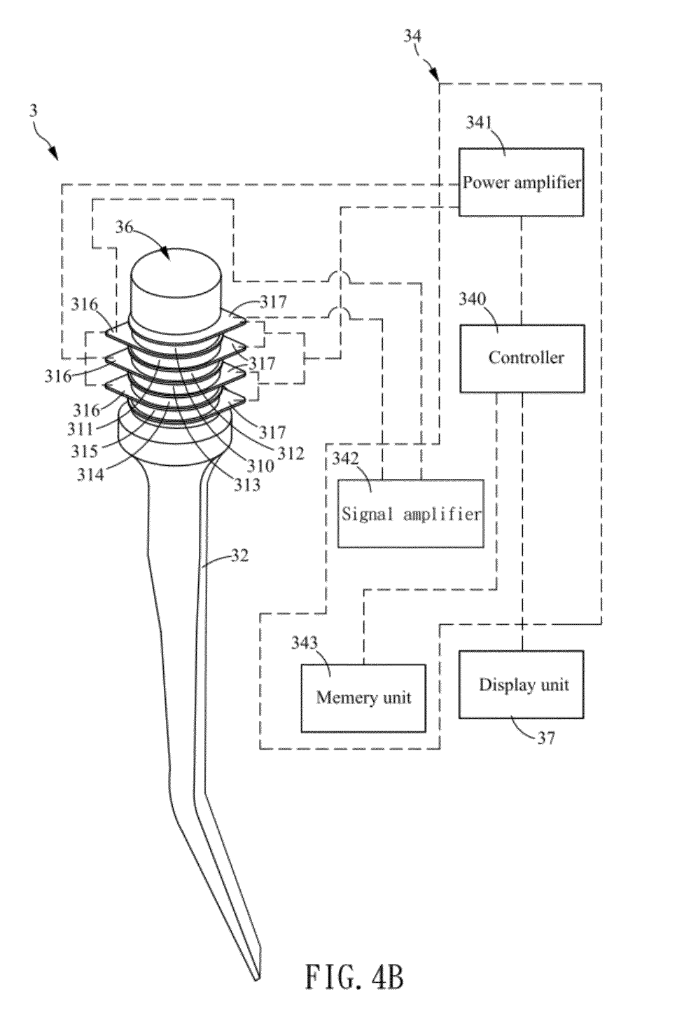

Please refer to FIG. 4A, which is a schematic diagram showing a tissue treatment device according to another embodiment of the present invention. The tissue treatment device shown in the embodiment of FIG. 4A is basically the same as the one shown in FIG. 2A, but is different in that: there is no insulation layer being configured in the tissue treatment device of FIG. 4A, and consequently, there is one anode plate 317 shared by the transducing elements 33 and 310 while arranged a cathode plate 331 at a side of the transducing element 33 that is opposite to the anode plate 317, whereas the anode plate 317 and the cathode plate 331 are then being electrically connected to the signal amplifier 342. Please refer to FIG. 4B, which is a schematic diagram showing a tissue treatment device according to further another embodiment of the present invention. Similarly, the tissue treatment device shown in the embodiment of FIG. 4B is basically the same as the one shown in FIG. 2A, but is different in that: there is no such vibration detection unit required to be configured in the tissue treatment device of FIG. 4B for detecting the second vibration signal, but instead, the second vibration signal resulting from the tissue feedback of the treatment tool 32 is captured by the use of at least one transducing element in the driving unit 31. IN FIG. 4B, although the signal amplifier 342 is coupled to the anode plate 317 and cathode plate 316 that are dispose respectively at the two sides of the topmost transducing element 310, the required second vibration signal can be captured using other single transducing element or any one or more than one of the plural transducing elements 310˜315.

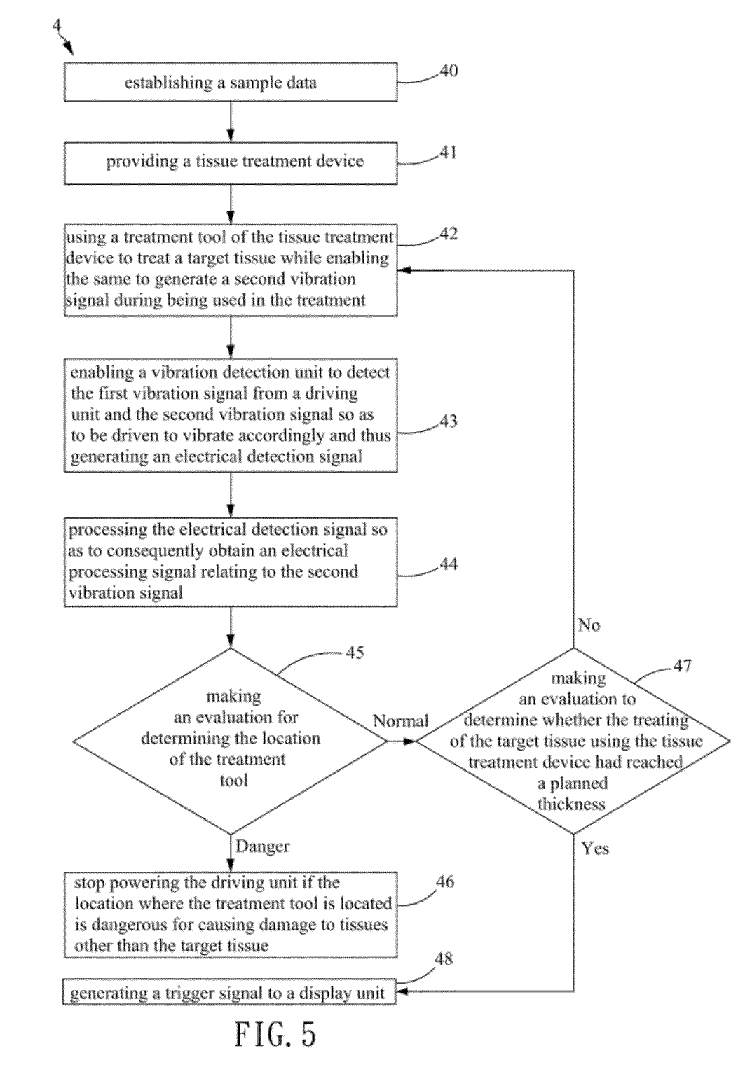

Please refer to FIG. 5, which is a flow chart depicting the steps performed in a signal processing method for tissue treatment device according to the present invention. The flow starts from the step 40. At step 40, a sample data is established so as to construct a database containing the second vibration signals that are generated in response to the different feedbacks corresponding to the different tissues and their thicknesses that are being treated by the treatment tool; and then the flow proceeds to step 41. It is noted that the tissues mentioned in the step 40 includes hard tissues, such as bones and teeth, and soft tissues, such as sinus membrane in the sinus cavity, oral call in mouth cavity, pulp cavity in any teeth, bone marrow in any bones, muscles surrounding any bones or internal organs. At step 41, a tissue treatment device 3, such as the one shown in FIG. 2A and FIG. 2B, or the one shown in FIG. 4A and FIG. 4B, is provided, which contained a treatment tool 32 capable of being driven to vibrate according to a first vibration signal issued from a driving unit 32; and then the flow proceeds to step 42. At step 42, the treatment tool 32 is used for treating a target tissue, and thus generating a second vibration signal during the treating of the target tissue; and then the flow proceeds to step 43. It is noted that, during the treating of the target tissue using the treatment tool 32, it is possible that the treatment tool might accidentally engage with tissues other than the target tissue, which can be soft tissues, such as sinus membrane, gums, muscle or internal organs, or hard tissues, such as bone and teeth, and in detail, each tooth can further be composed of different tissues including enamel, dentin and pulp cavity, and thus, the second vibration signal generated from the treatment tool 32 can be varied with the variation of the tissues that is being treated by the treatment tool 32 as the feedbacks corresponding to the different tissues that are being treated by the treatment tool 32 are different. i.e. the treatment tool 32 can generated different second vibration signals corresponding to different feedbacks resulting from the engaging of the treatment tool 32 with different tissues.

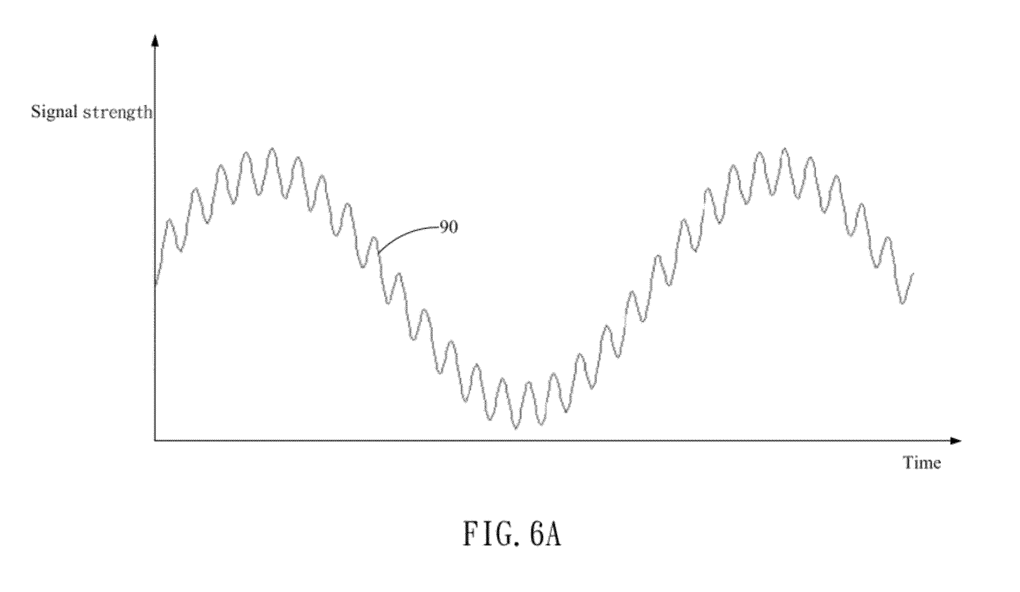

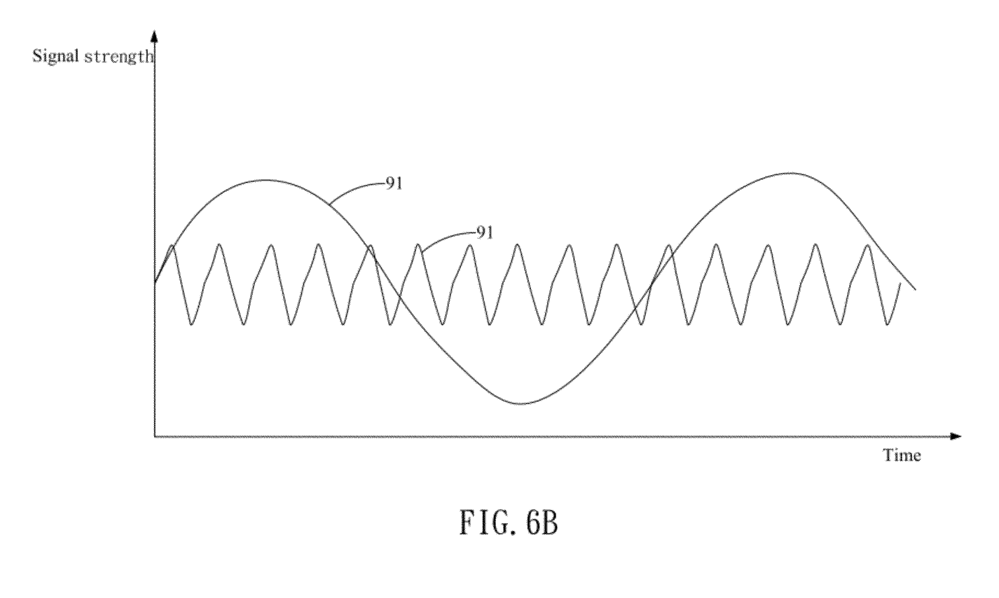

At step 43, a vibration detection unit 33, being arranged coupling to the driving unit 31 and the treatment tool 32, is enabled to detecting the first vibration signal and the second vibration signal so as to be driven to vibrate accordingly and thus generating an electrical detection signal; and then the flow proceeds to step 44. It is noted that the vibration detection unit 33 in the present embodiment is substantially a transducing element, so that it is capable of generating the electrical detection signal in response to the vibration induced by the first and the second vibration signals in a manner shown in FIG. 6A. As shown in FIG. 6A, the electrical detection signal 90 is the combination of the electrical signal induced by the first vibration signal and the electrical signal induced by the second vibration signal. Thereafter, at step 44, the electrical detection signal is processed so as to consequently obtain an electrical processing signal relating to the second vibration signal; and then the flow proceeds to step 45. Moreover, as the electrical detection signal 90 is the combined electric signals relating to the first and the second vibration signals, the electrical detection signal of FIG. 6A can be separated by the use of a filtering means into the two signals shown in FIG. 6B, in which the curve 91 represents the electrical processing signal relating to the first vibration signal, and the curve 92 represents the electrical processing signal relating to the second vibration signal.

At step 45, the electrical processing signal obtained from step 44 is compared with the sample data established in step 41 so as to determine the location of the treatment tool and thus determine whether the treatment tool is located at a position that is dangerous for causing damage to tissues other than the target tissue, or is located at a position that is considered normal for treating the target tissue; and if the treatment tool is determined to be located at a dangerous position, the flow will proceed to step 46, otherwise the flow will proceed to step 47 when the treatment tool is determined to be located at a normal position. It is noted that the so-called dangerous position can be the soft tissue area relating to gums or pulp cavity when the treatment tool is used for treating a tooth. However, in the condition when the treatment tool is used for treating an alveolar bone, the dangerous position is the area near the sinus membrane as shown in FIG. 3. In addition, in the condition when the treatment tool is used for treating a hard tissue, such as bones, the dangerous position is the muscles or internal organs surrounding the treated bone, or the bone marrow of the treated bone. On the other hand, the so-called normal position will be the enamel part or the dentin part when the treatment tool is used for treating a tooth, or can be the alveolar bone, the compact bone, and the sponge bone when the treatment tool is used for treating bones. At step 46, as soon as the treatment tool is determined to be located at a dangerous position, the power of the driving unit will be stopped immediately for stopping the activation of the driving unit. At step 47, as soon as the treatment tool is determined to be located at a normal position, the controller 34 is enabled to make an evaluation based upon the comparison between the electrical processing signal and the sample data for determining whether the treating of the target tissue using the treatment tool had reached a planned thickness; and if so, the flow proceeds to step 48; otherwise, the flow proceeds back to step 42. At step 48 that the controlled had reach a conclusion that the planned thickness of the target tissue had already been reached, the controller 340 is enabled to generate a trigger signal to a display unit 37 for enabling the same to generate and display an alert signal to the physician operating the treatment tool 32 and thus informing the physician that the specific thickness of the target tissue had already been reached.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

What is claimed is:

1. A tissue treatment device, comprising:
a driving unit, for generating a first vibration signal;
a treatment tool, coupled to the driving unit, for receiving the first vibration signal to be used for enabling the same to vibrate while generating a second vibration signal during being used in a treatment;
a vibration detection unit, for detecting the first vibration signal and the second vibration signal so as to be driven to vibrate accordingly and thus generating an electrical detection signal; and
a signal processing unit, coupled to the vibration detection unit for enabling the same to receive and process the electrical detection signal and thus obtain an electrical processing signal relating to the second vibration signal.

2. The tissue treatment device of claim 1, wherein the driving unit is composed of a plurality of transducing elements that are stacking on each other.

3. The tissue treatment device of claim 1, wherein the vibration detection unit is substantially a transducing element.

4. The tissue treatment device of claim 1, further comprising:
an insulation layer, sandwiched between the vibration detection unit and the driving unit.

5. The tissue treatment device of claim 1, wherein the signal processing unit further comprises:
a signal amplifier, coupled to the vibration detection unit to be used for amplifying the electrical detection signal; and
a controller, coupled to the signal amplifier to be used for processing the electrical detection signal so as to obtain the electrical processing signal.

6. The tissue treatment device of claim 5, wherein the signal processing unit further comprises:
a memory unit, for storing a sample data relating to all kinds of tissues while providing the sample data to the controller to be compared with the electrical processing signal so as to determine the location of the treatment tool based upon the comparison.

7. The tissue treatment device of claim 6, further comprises:
a display unit, coupled to the controller.

8. A tissue treatment device, comprising:
a treatment tool, for receiving a first vibration signal to be used for enabling the same to vibrate while generating a second vibration signal during being used in a treatment;
a driving unit, composed of a plurality of transducing elements that are stacking on each other, for generating the first vibration signal and for receiving the second vibration signal so as to enable at least one of the plural transducing elements to generate an electrical detection signal according to the received second vibration signal; and
a signal processing unit, coupled to at least one of the plural transducing elements of the driving unit for enabling the same to process the electrical detection signal and thus obtain an electrical processing signal relating to the second vibration signal.

9. The tissue treatment device of claim 8, wherein the signal processing unit further comprises:
a signal amplifier, coupled to at least one of the plural transducing elements so as to be used for amplifying the electrical detection signal; and
a controller, coupled to the signal amplifier to be used for processing the electrical detection signal so as to obtain the electrical processing signal.

10. The tissue treatment device of claim 9, wherein the signal processing unit further comprises:
a memory unit, for storing a sample data relating to all kinds of tissues while providing the sample data to the controller to be compared with the electrical processing signal so as to determine the location of the treatment tool based upon the comparison.

11. The tissue treatment device of claim 9, further comprising:
a display unit, coupled to the controller.

12. A signal processing method for tissue treatment device, comprising the steps of:
providing a tissue treatment device configured with a driving unit and a treatment tool in a manner that the driving unit is used for generating a first vibration signal to be used for controlling the treatment tool to vibrate accordingly;
applying the treatment tool to treat a target tissue while enabling the same to generate a second vibration signal to be generated during the treating of the target tissue;
generating an electrical detection signal according to the vibrations resulting from the first vibration signal and the second vibration signal; and
processing the electrical detection signal so as to consequently obtain an electrical processing signal relating to the second vibration signal.

13. The signal processing method of claim 12, further comprising the steps of:
comparing the electrical processing signal with at least one sample data so as to determine the location of the treatment tool based upon the comparison; and
generating a display signal if the determined location is the location of the target tissue

14. The signal processing method of claim 13, wherein the display signal is a signal selected from the group consisting of: a visual signal and an audio signal.

15. The signal processing method of claim 13, further comprising the step of:
stop powering the driving unit if the location where the treatment tool is located is dangerous for causing damage to tissues other than the target tissue.

16. The signal processing method of claim 12, further comprising the steps of:
amplifying the electrical detection signal; and
filtering the electrical detection signal so as to obtain the electrical processing signal relating to the second vibration signal.
